(19) United States
(12) Patent Application Publication
(10) Pub. No.: US 2017/0291017 A1
(43) Pub. Date: Oct. 12, 2017

(54) SUBDURAL DRAINAGE CATHETER WITH SELF CONTAINED MECHANISM FOR RESTORATION OF FLOW FOLLOWING CATHETER OBSTRUCTION

(71) Applicant: Wake Forest University Health Sciences, Winston-Salem, NC (US)

(72) Inventors: Charles L. Branch, JR., Advance, NC (US); John P. Eickman, Winston-Salem, NC (US)

(21) Appl. No.: 15/511,475

(22) PCT Filed: Sep. 23, 2015
(86) PCT No.: PCT/US2015/051679

§ 371 Date: Mar. 15, 2017

Related U.S. Application Data
(60) Provisional application No. 62/053,964, filed on Sep. 23, 2014.

(51) Int. Cl. A61M 27/00; A61M 39/02; A61M 39/04; A61M 25/00

(52) U.S. Cl. CPC A61M 27/00; A61M 25/0017; A61M 25/002; A61M 39/0247; A61M 39/04; A61M 2025/0019; A61M 2039/025; A61M 2039/0264; A61M 2039/0273; A61M 2039/0276; A61M 2205/0266

(57) ABSTRACT

A subdural drainage device includes a subdural drainage housing defining a drainage passageway. The drainage passageway has a A subdural drainage device includes a subdural drainage housing defining a drainage passageway. The drainage passageway has a lower opening configured to connect to a subdural space of a patient and an upper opening. A blockage removal unit is in the housing, and the blockage removal unit has an end portion that is configured to reduce or remove blockages adjacent the lower opening of the drainage passageway that is movable between a retracted position in which the end portion is in the subdural drainage housing and an extended position in which the end portion of the blockage removal unit extends away from the subdural drainage housing via the lower opening of the drainage passageway. At least one side delivery port is in the housing and in fluid communication with the drainage passageway.

### RELATED APPLICATIONS

This application claims priority to U.S. Provisional Application 62/053,964, filed Sep. 23, 2014, and is related to PCT Application No. PCT/US2014/031441, filed Mar. 21, 2014, which claims priority to U.S. Provisional Application Ser. No. 61/803,952, filed Mar. 21, 2013, and is related to U.S. application Ser. No. 13/848,285, filed Mar. 21, 2013, which is a continuation of U.S. application Ser. No. 13/130,238, filed Aug. 2, 2011, which will issue as U.S. Pat. No. 8,403,878 on Mar. 26, 2013, and is a 35 U.S.C. §371 national phase application of PCT Application PCT/US2009/065282, filed Nov. 20, 2009, and published in English on May 27, 2010, as International Publication No. WO 2010/059915 A1, and which claims the benefit under 35 U.S.C. §119(e) of U.S. Provisional Patent Application Ser. No. 61/116,525, filed Nov. 20, 2008, the disclosure of each of which is incorporated herein by reference in its entirety.

### FIELD OF THE INVENTION

The present invention concerns medical devices in general, and particularly concerns devices for the treatment of subdural hematoma.

### BACKGROUND OF THE INVENTION

Subdural hematomas continue to be a challenging set of pathology for neurosurgeons given today's aging population. Patients with symptomatic subacute and chronic subdural hematomas are frequently elderly and commonly have multiple coexisting medical conditions. Typically patients present after a remote history of trauma, though often trivial, with progressive neurologic deficits including hemiparesis, gait instability, and cognitive decline to name a few. Ramachandran R, Thimmappa H. “Chronic subdural hematomas—causes of morbidity and mortality.” Surg Neurol 2007:67;367-373. The mortality rate for this pathology even with treatment, often cited at a range of 2-6%, is significant given the frequency of symptomatic presentation. Sucu H K, Gokmen M, Ergin A, Bezircioglu, Gokemn A. “Is there a way to avoid surgical complications of twist drill craniostomy for evacuation of a chronic subdural hematoma.” Acta Neurochir (Wien) 2007;149:597-599; Ernestus R I, Beldzinski P, Lanfermann H, Klug N. “Chronic subdural hematoma: Surgical treatment and outcome in 104 patients.” Surg Neurol 1997;48:220-225; Mellergard P, Wisten O. “Operations and re-operations for chronic subdural haematomas during a 25-year period in a well defined population.” Acta Neurochir (Wien) 1996; 138:708-713; and Merlicco G, Pierangeli E, di Padova P L. “Chronic subdural hematomas in adults: prognostic factors.” Analysis of 70 cases. Neurosurg Rev 1995;18:247-251. Multiple treatment modalities have been described and advocated in the literature. The most extreme is a standard craniotomy under general anesthesia for hematoma evacuation and membrane removal, if present. Alternatively, isolated or multiple bur holes may be drilled to permit hematoma irrigation and removal. More recently, placement of a tangential drainage catheter within the subdural space via a twist-drill craniostomy had been practiced typically under monitored sedation, but catheter obstruction and cessation of flow is often encountered.

A subdural evacuation port system (or “SEPS”) available from Medtronic, Inc., Minneapolis, Minn., has seen recent use. As illustrated in FIGS. 1a-1b, a SEPS device 10 is essentially a hollow screw placed in the skull 20 which communicates with the subdural space 22 and is attached to closed system suction device (not shown). Asfora WT, Schwebach L. “A modified technique to treat chronic and subacute subdural hematoma: technical report.” Surg Neurol 2003;59:329-332. While the device 10 has appeared more successful that the tangential subdural catheter technique and can be performed without general anesthesia, it is potentially complicated by portal obstruction prior to adequate hematoma evacuation.

Indeed, recent experience of one surgical group with the SEPS device indicates that approximately one third of patients treated had less than half of the hematoma volume evacuated. This subsequently resulted in approximately one quarter of the patients requiring an additional surgical procedure to adequately evacuate the hematoma due to inability to restore drainage of fluid through this system after it became occluded. One could open the system to clear occlusions by insert a trochar, but this potentially breaks sterility, and insertion of a trochar or other tool raises the potential of injuring underlying brain tissue. Hence, there is a need for new devices for the treatment of subdural hematoma.

### SUMMARY OF THE INVENTION

According to some embodiments of the present invention, a subdural drainage device includes a subdural drainage housing defining a drainage passageway. The drainage passageway has a lower opening configured to connect to a subdural space of a patient and an upper opening. A blockage removal unit is in the housing, and the blockage removal unit has an end portion that is configured to reduce or remove blockages adjacent the lower opening of the drainage passageway that is movable between a retracted position in which the end portion is in the subdural drainage housing and an extended position in which the end portion of the blockage removal unit extends away from the subdural drainage housing via the lower opening of the drainage passageway. At least one side delivery port is in the housing and in fluid communication with the drainage passageway. The at least one side delivery port is configured to receive a delivery device so that the delivery device delivers a therapeutic agent to the drainage passageway via the at least one side delivery port.

In some embodiments, the side delivery port comprises a port passageway having an outer opening that is configured to receive a delivery device and an inner opening that connects to the drainage passageway. The outer opening may include a resealable member configured to be punctured by a delivery device comprising a needle and to be resealed when the needle is removed from the resealable member.

In some embodiments, the end portion of the blockage removal unit that is configured to reduce or remove blockages comprises a deformable member that is configured to move between a compact configuration when the end portion is in the subdural drainage housing in the retracted position and an expanded position when the end portion extends away from the subdural drainage housing via the lower opening of the drainage passageway in the extended position.

In some embodiments, the blockage removal unit is coaxial with the drainage passageway. The blockage removal unit may include a drainage passageway therethrough. The blockage removal unit may include a biasing member configured to maintain the blockage removal unit in the retracted position. In some embodiments, a locking member is configured to move between a locked position in which a portion of the locking member abuts a portion of the blockage removal unit to maintain the blockage removal unit in the retracted position and an unlocked position in which the locking member releases the blockage removal unit so that the blockage removal unit may be moved to the extended position.

In some embodiments, the deformable member has a diameter that is greater than a diameter of the drainage passageway when the deformable member is in the extended position.

In some embodiments, the subdural drainage housing provides a sealed enclosure when the subdural drainage housing is connected to a skull of a subject.

In some embodiments, the blockage removal unit is mounted in a side of the housing and in communication with the drainage passageway.

In some embodiments, first and second sealing members are in the drainage passageway defining a sealing chamber. The first sealing member may be mounted on the housing, and the second sealing member may be mounted on the blockage removal unit such that when the second sealing member moves toward the first sealing member when the blockage removal unit is moved from the retracted position to the extended position to thereby increase or maintain sterility of the fluid drainage passageway.

In some embodiments, a kit comprises the device described above. The device may be sealed in one or more a sterile packages or containers.

In some embodiments, a method for draining the subdural space includes positioning a subdural drainage system on a patient, the subdural drainage system comprising: a subdural drainage housing defining a drainage passageway, the drainage passageway having a lower opening configured to connect to a subdural space of a patient and an upper opening; a blockage removal unit in the housing, the blockage removal unit having an end portion that is configured to reduce or remove blockages adjacent the lower opening of the drainage passageway and being movable between a retracted position in which the end portion is in the subdural drainage housing and an extended position in which the end portion of the blockage removal unit extends away from the subdural drainage housing via the lower opening of the drainage passageway; and at least one side delivery port in the housing and in fluid communication with the drainage passageway, the at least one side delivery port being configured to receive a delivery device so that the delivery device delivers a therapeutic agent to the drainage passageway via the at least one side delivery port. The method includes moving the blockage removal unit between the retracted and extended positions so that the deformable member automatically extends to an extended, deformed position in which blockages are disrupted in and adjacent the drainage passageway.

The present invention is explained in greater detail in the drawings herein and the specification set forth below. All United States Patent references cited herein are to be incorporated by reference herein in their entirety.

### BRIEF DESCRIPTION OF THE DRAWINGS

FIG. 1A (Comparative) is a subdural evacuating port system (SEPS) device.

FIG. 1B (Comparative) is the subdural evacuation port system (SEPS) device of FIG. 1A positioned such that a threaded portion thereof is twisted into the skull and the notched ended is attached to silastic tubing connected to a self-suction bulb.

FIG. 2 is a perspective view of a subdural drainage device with a drug delivery port according to some embodiments.

FIGS. 3-10 are perspective cut away views of the subdural drainage device of FIG. 2 in a retracted position (FIGS. 3, 5, 7 and 9) and an extended position (FIGS. 4, 6, 8 and 10).

FIGS. 11-12 are exploded views of the deformable member of the device of FIG. 2 in the retracted position (FIG. 11) and the extended position (FIG. 12)

FIGS. 13-14 are perspective views of a blockage removal unit of the device of FIG. 2.

FIG. 15 is a side perspective cross sectional view of the device of FIG. 1.

FIGS. 16-17 are exploded views of a locking member of the device of FIG. 1.

FIG. 18 is a perspective view of a subdural drainage device according to some embodiments.

FIG. 19 is a perspective cut-away view of the subdural drainage device of FIG. 18.

FIGS. 20-21 are perspective cross-sectional views of the subdural drainage device of FIG. 18 in a retracted position (FIG. 20) and an extended position (FIG. 21).

FIG. 22 is an exploded view of the device of FIG. 18.

FIGS. 23-25 are perspective views of a subdural drainage device according to some embodiments.

FIG. 26 is a cross sectional view of the device of FIGS. 23-25.

FIG. 27 is a cut-away view of the device of FIGS. 23-26.

FIG. 28 is a cut-away view of the device of FIGS. 23-27 with the deformable member in the retracted position.

FIG. 29 is a cut-away view of the device of FIGS. 23-28 with the deformable member in the extended position.

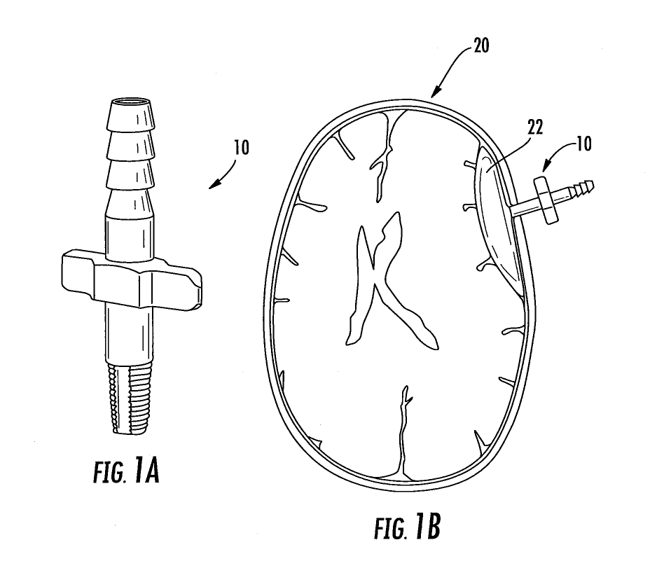

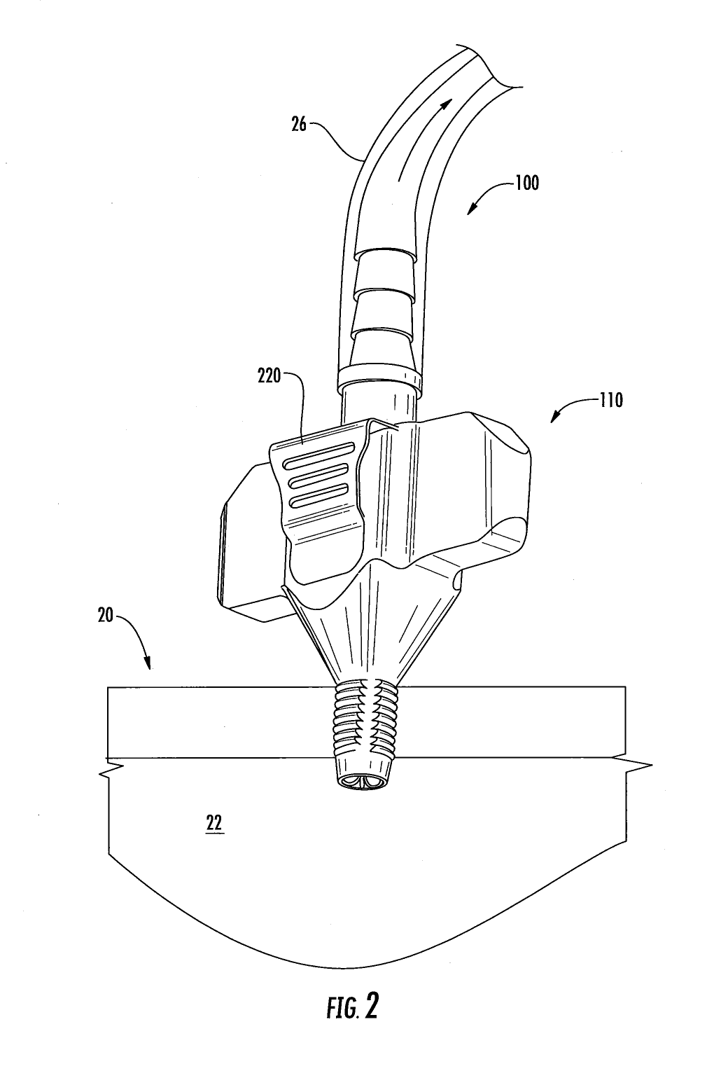

### DETAILED DESCRIPTION OF EMBODIMENTS OF THE INVENTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

In some embodiments, a subdural drainage housing defines a drainage passageway that has a lower opening that is configured to connect to a subdural space of a patient and an upper opening that can be connected to a drainage reservoir via a tube or other fluid conduit. A blockage removal unit is in the housing that has an end portion that is configured to reduce or remove blockages adjacent the lower opening of the drainage passageway and is movable between a retracted position (in which the end portion is in the subdural drainage housing) and an extended position (in which the end portion of the blockage removal unit extends away from the subdural drainage housing via the lower opening of the drainage passageway. A side delivery port is in the housing and is in fluid communication with the drainage passageway. The side delivery port is configured to receive a delivery device, such as a device for delivering a drug or other therapeutic agent, so that the delivery device delivers the therapeutic agent to the drainage passageway via the side port.

Thus, a sealed housing may provide a sealed enclosure when subdural drainage device is in position on a subject patient. Obstructions may be cleared and/or drugs may be delivered without requiring removal of a subdural drainage device.

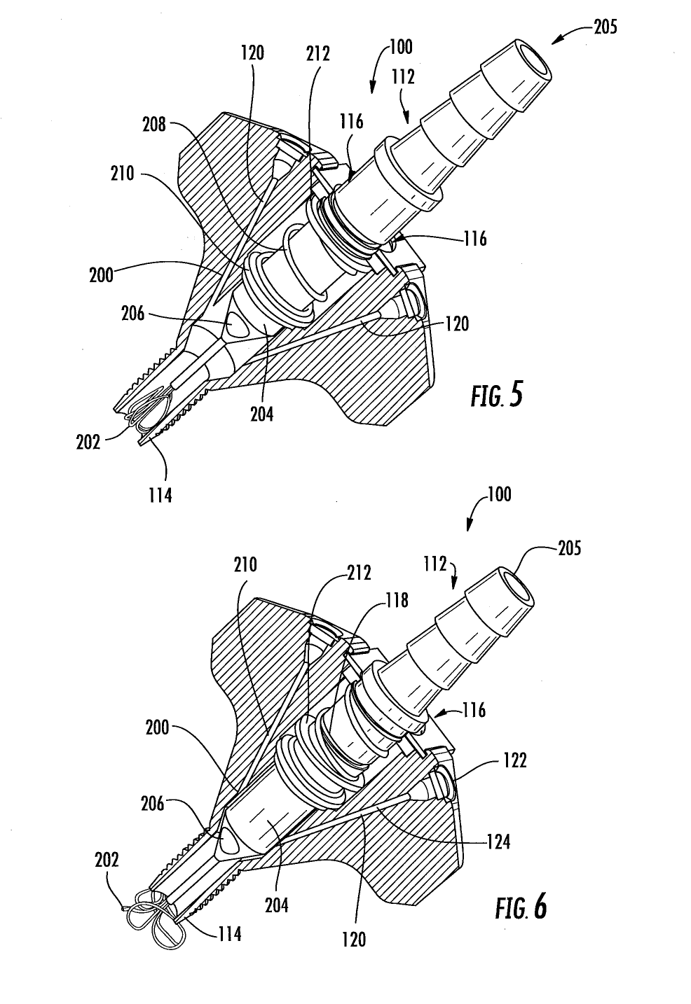

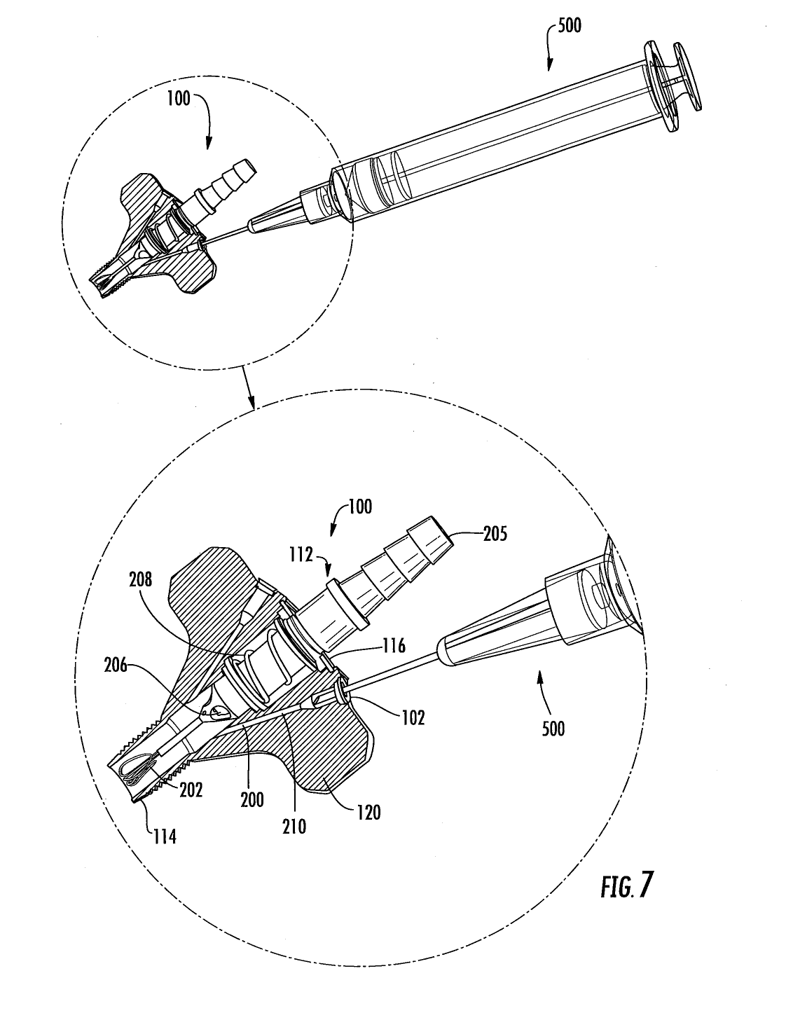

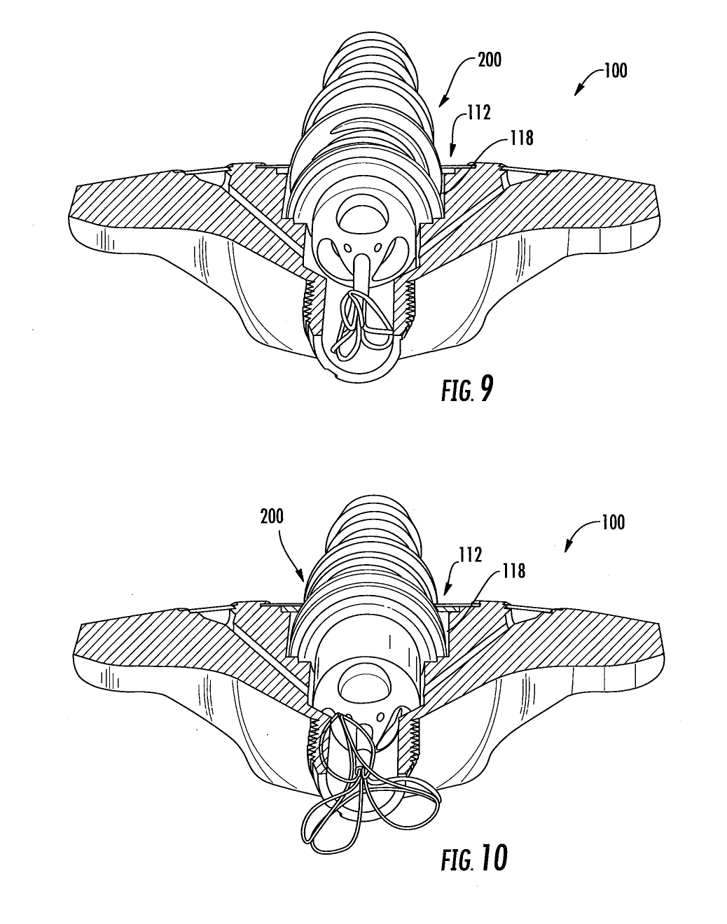

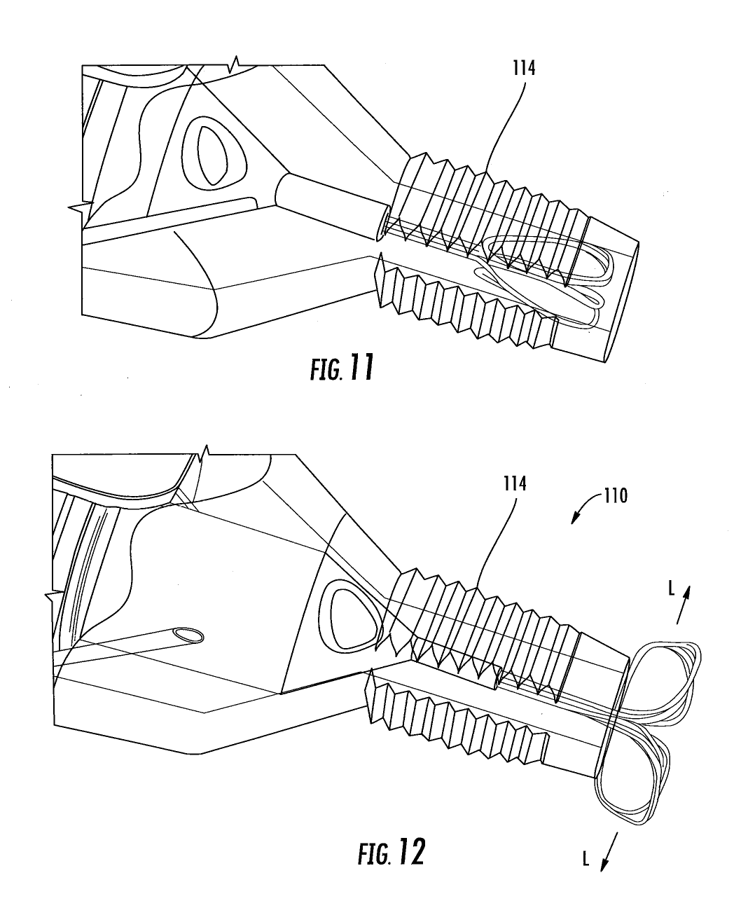

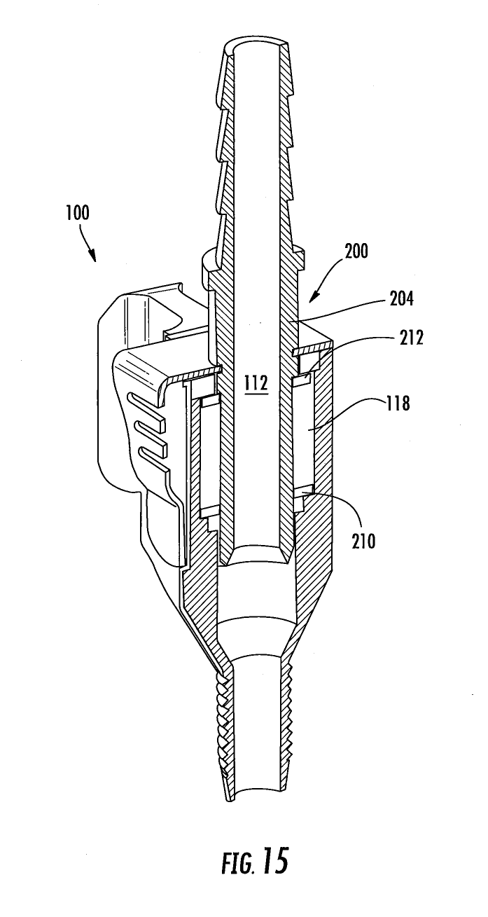

As illustrated in FIGS. 2-17, a subdural drainage device 100 is shown. The subdural drainage device 100 includes a housing 110 that has a drainage passageway 112 with a lower opening 114 configured to connect to a subdural space 22 of a skull 20, and an upper opening 116. The housing 110 includes a sealing chamber 118 in the drainage passageway 112. The device 100 further includes a blockage removal unit 200 in the housing 110 and two side delivery ports 120. The side delivery ports 120 include a sealing member 122 and a conduit 124.

The blockage removal unit 200 includes an end portion or deformable member 202 and a body 204. The body 204 is generally hollow and includes apertures 206 for receiving fluid from the housing opening 114. The body 204 further includes an end 205 that is configured to connect the housing drainage passageway 112 to a tube 26 (FIG. 2). The unit 200 includes a biasing member 208, two sealing members 210, 212 and a locking member 220. The locking member 220 is mounted on the housing 110 and includes an aperture 222 that receives the end 205.

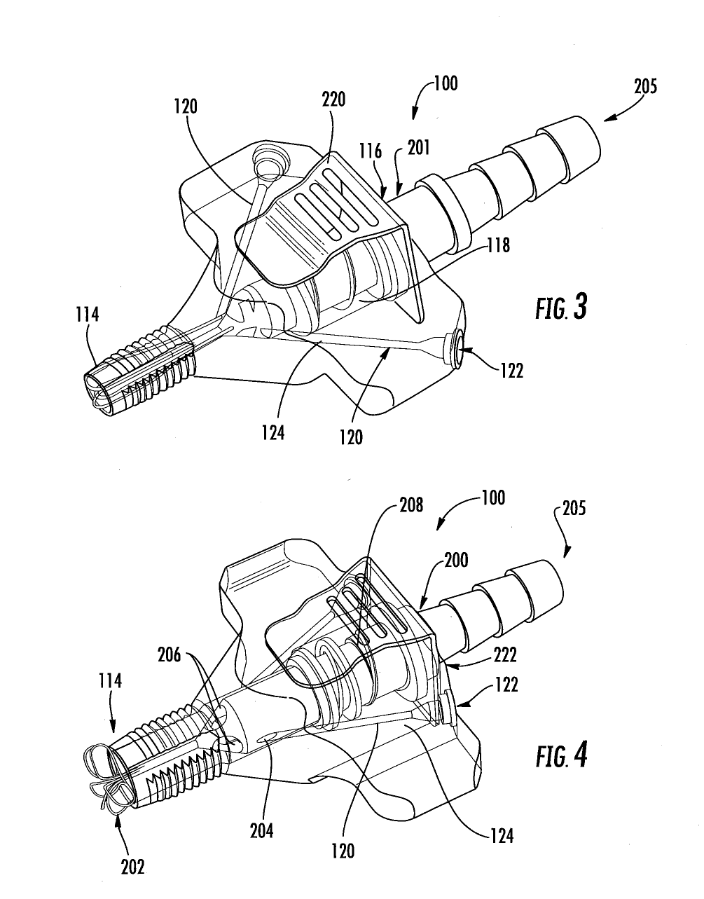

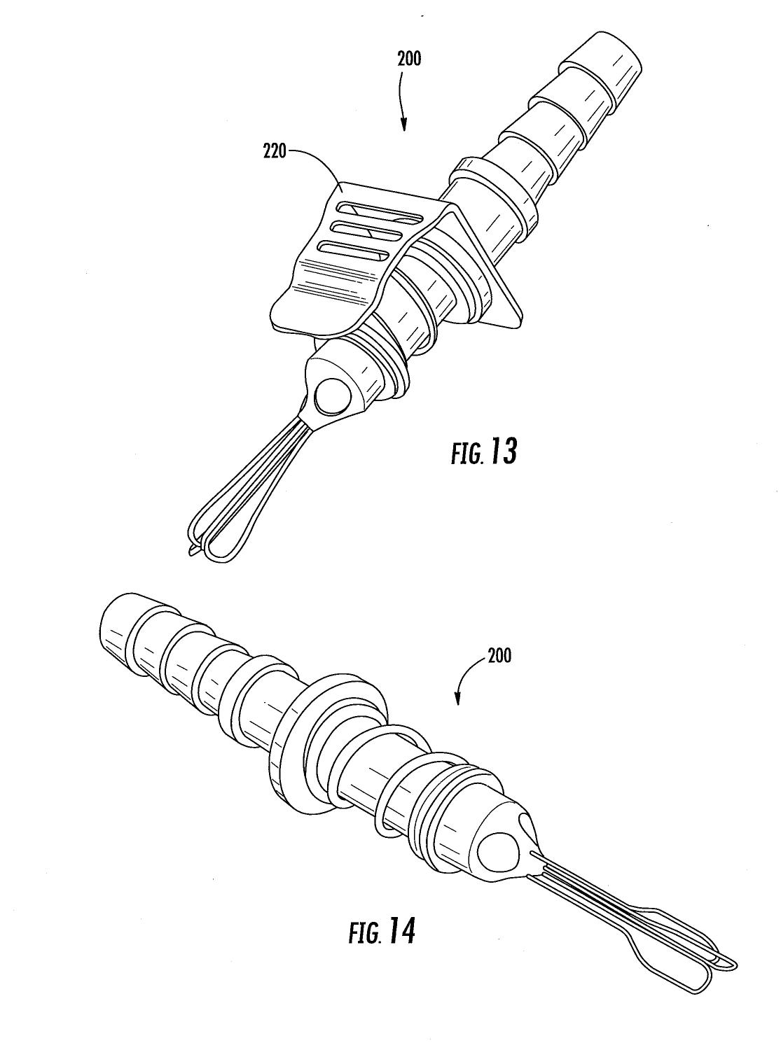

As illustrated in FIGS. 3-12, The blockage removal unit 200 is movable between a retracted position (FIGS. 3, 5, 7, 9 and 11) in which the deformable member 202 is in the housing 110 and an extended position (FIGS. 4, 6, 8, 10 and 12) in which the deformable member 202 extends away from the housing 110 via the lower opening 114. As illustrated in FIG. 12, the deformable member 202 extends in a lateral direction L with respect to the opening 114. In this configuration, the deformable member 202 is configured to dislodge, reduce and/or remove blockages that may accumulate adjacent the opening 114 in the subdural space 22 (FIG. 2). The deformable member 202 may extend further from the edges of the opening 114 without extending too deeply along the longitudinal axis of the housing 110 into the subdural space 22. The blockage removal unit 200 may also be configured to rotate around its lateral axis so that the deformable member 202 may rotate and further dislodge any blockages in the subdural space 22. In some embodiments, the deformable member 202 is formed of a shape memory material. Any suitable shape memory material may be used, including shape memory alloys such as nitinol, shape memory polymers, and composites thereof (with other shape memory or non-shape memory materials to form a shape-memory composite material or structure). See, e.g., U.S. Pat. Nos. 8,328,842 and 7,879,004; C. Ykacki et al., Unconstrained Recovery Characterization of Shape-Polymer Networks for Cardiovascular Applications, Biomaterials 28, 2255-2263 (2007); see generally K. Otsuka and C. Wayman, Shape Memory Materials (Cambridge University Press 1999).

As illustrated, the body 204 of the blockage removal unit 200 forms a generally hollow fluid chamber such that the fluid from the subdural space 22 is permitted to flow into the housing 110 from the opening 114 and through the apertures 206 into the body 204 of the unit 200 where it exits the body 204 at the end 205. In this configuration, the fluid flow from the subdural space 22 is generally unobstructed during deployment of the deformable member 202.

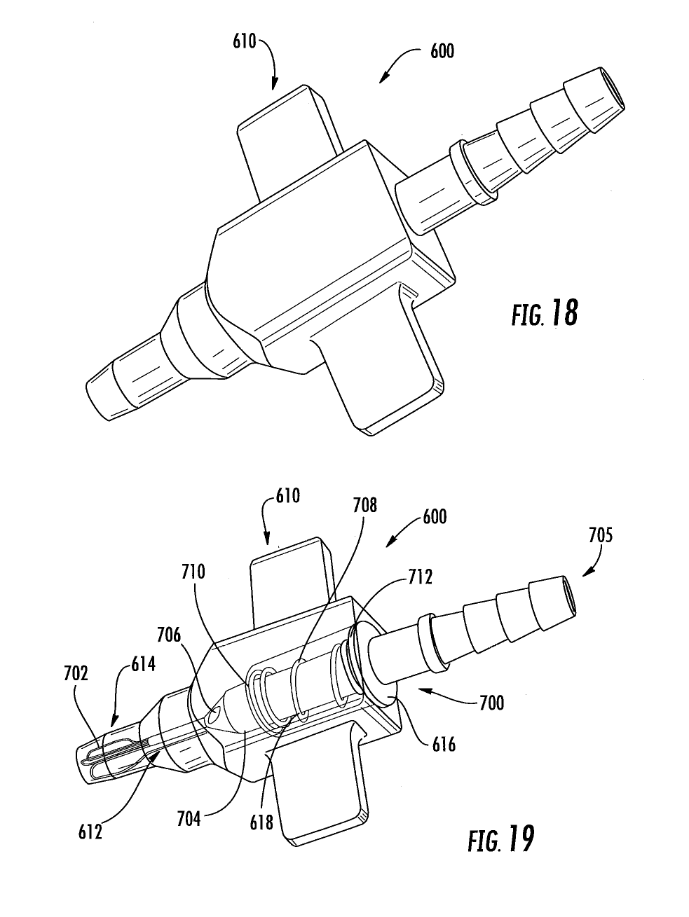

As illustrated in FIGS. 16-17, the locking member 220 abuts an edge or ridge portion of the body 204 at the locking member aperture 220 to lock or prevent movement of the blockage removal unit 200 in the lateral direction (FIG. 16). The locking member 220 may be moved by the user to an open position (FIG. 17) so that the aperture 220 allows free movement of the blockage removal unit 200 in the lateral direction. In the open position of FIG. 17, the spring or biasing member 208 biases the blockage removal unit 200 in the retracted position in the absence of pressure or force on the end 205. When a force is applied to the end 205, the blockage removal unit 200 is moved from the retracted position (FIGS. 3, 5, 7, 9 and 11) in which the deformable member 202 is in the housing 110 to the extended position (FIGS. 4, 6, 8, 10 and 12) in which the deformable member 202 is deployed in the subdural space. In this configuration, the deformable member 202 is generally prevented from being accidentally deployed into the subdural space, and once deployed, the deformable member 202 generally returns to the retracted position when the end 205 is released.

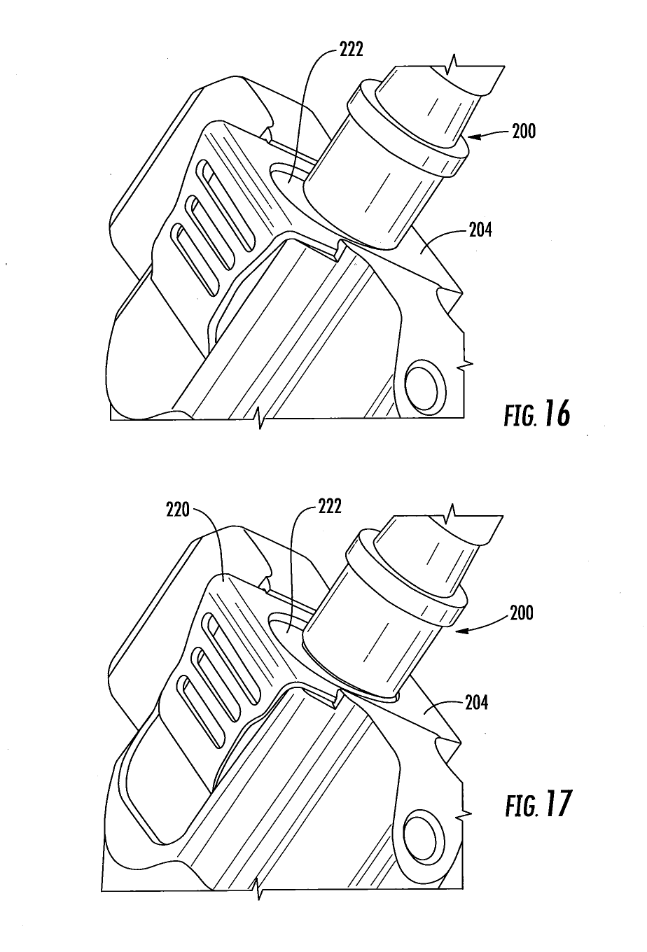

As shown in FIGS. 2-10 and in FIG. 15, the sealing chamber 118 and sealing members 210, 212 may maintain a hermetic seal and/or sterility of the subdural space 22. As illustrated, the O-ring or sealing member 210 is affixed to the housing 110 and is stationary with respect to the housing 110, and the sealing member 212 is affixed to the blockage removal unit 200 so that the sealing member 212 moves toward the sealing member 210 when the blockage removal unit 200 is moved from the retracted position to the extended position to thereby maintain sterility of the fluid drainage passageway 112 and the body 204 of the unit 200.

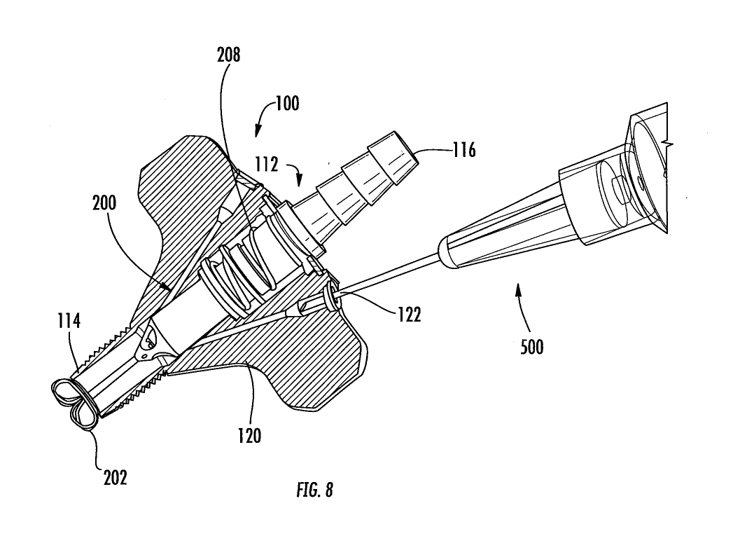

With reference to FIGS. 7-8, a drug delivery device or needle 500 is inserted in the drug delivery port 120 via the sealing member 122. The sealing member 122 may be a self-sealing plug formed of an elastomeric material that is configured to reseal after it is punctured with the needle 500. The needle 500 extends at least a portion of the way down the conduit 124 to dispense a therapeutic agent to the passageway 112 and into the subdural space 22. Any suitable drug or therapeutic agent may be used, including thrombolytic agents, antibiotic agents, and/or chemotherapy agents.

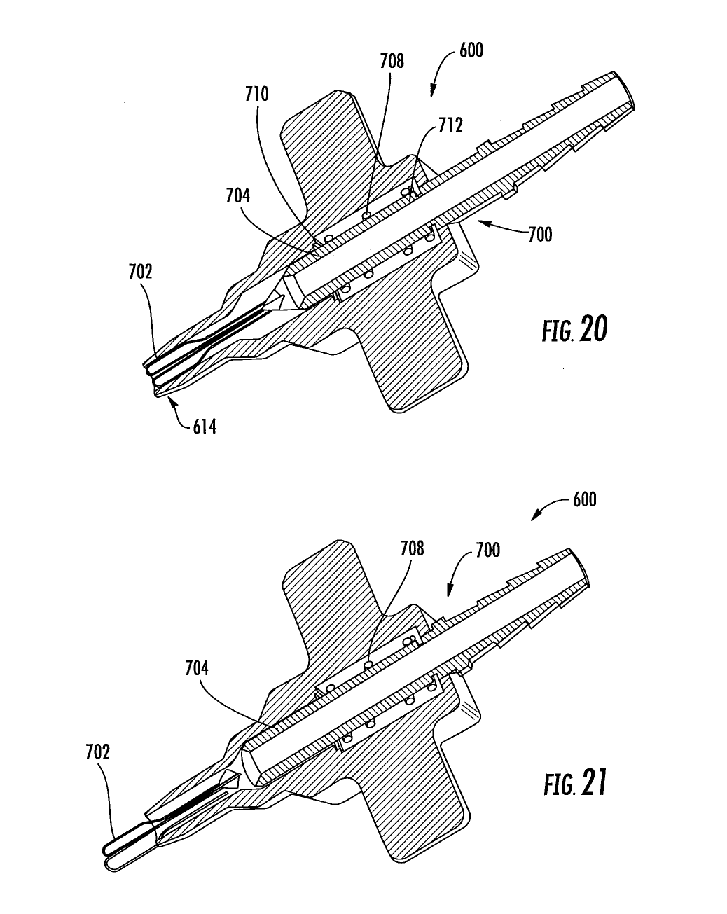

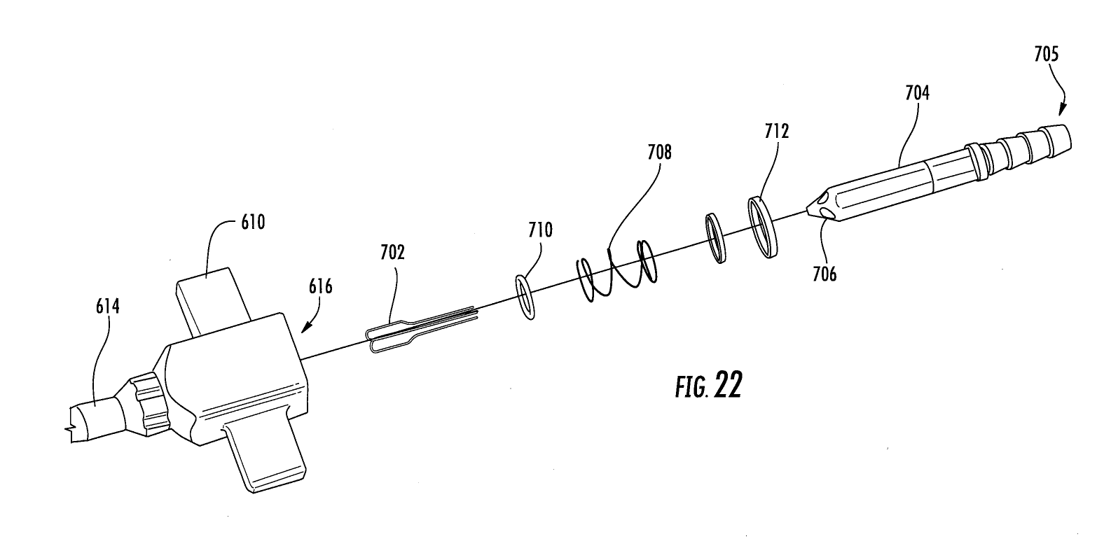

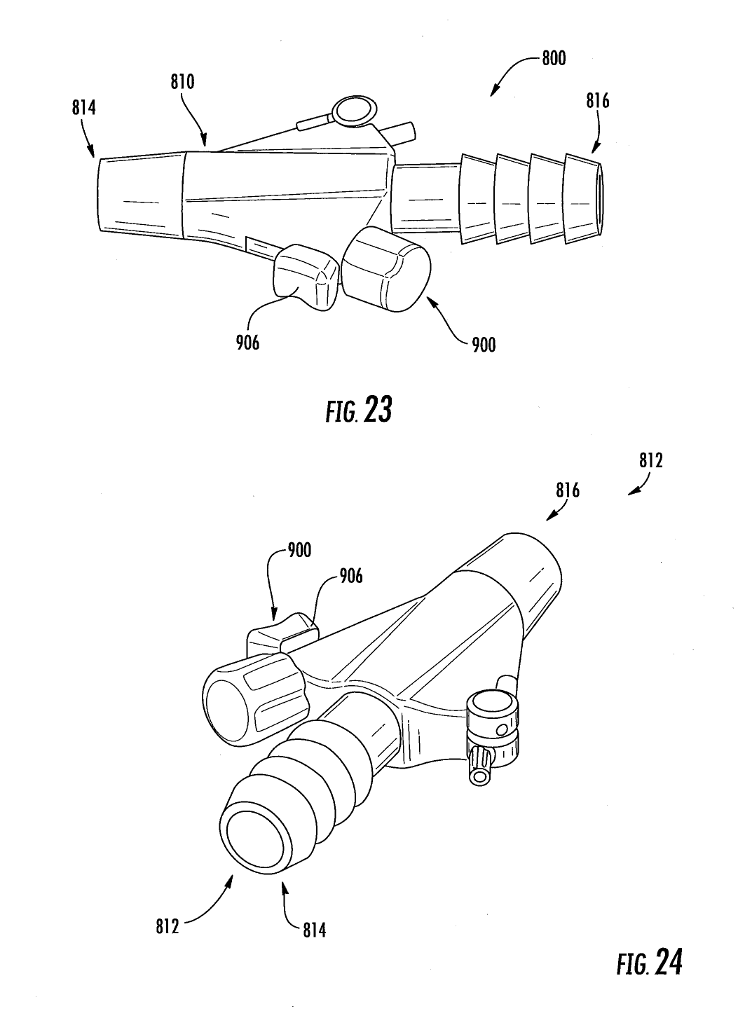

As shown in FIGS. 18-22, in some embodiments, subdural drainage device 600 omits the drug delivery ports. The subdural drainage device 600 includes a housing 610 that has a drainage passageway 612 with a lower opening 614 configured to connect to a subdural space 22 of a skull 20, and an upper opening 616. The housing 610 includes a sealing chamber 618 in the drainage passageway 612. A blockage removal unit 700 in the housing 610 and includes an end portion or deformable member 702 and a body 704. The body 704 is generally hollow and includes apertures 706 for receiving fluid from the housing opening 714. The body 704 further includes an end 705 that is configured to connect the housing drainage passageway 712 to a tube 76 (FIG. 2). The unit 700 includes a biasing member 708 and two sealing members 710, 712 that can maintain a hermetically sealed and/or sterile environment.

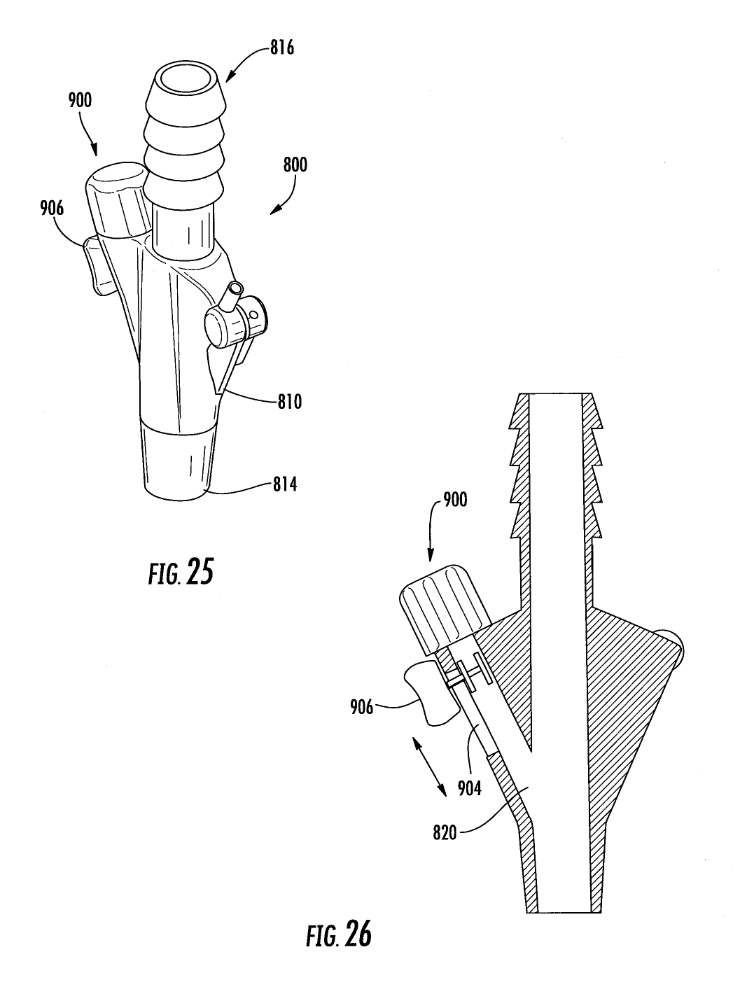

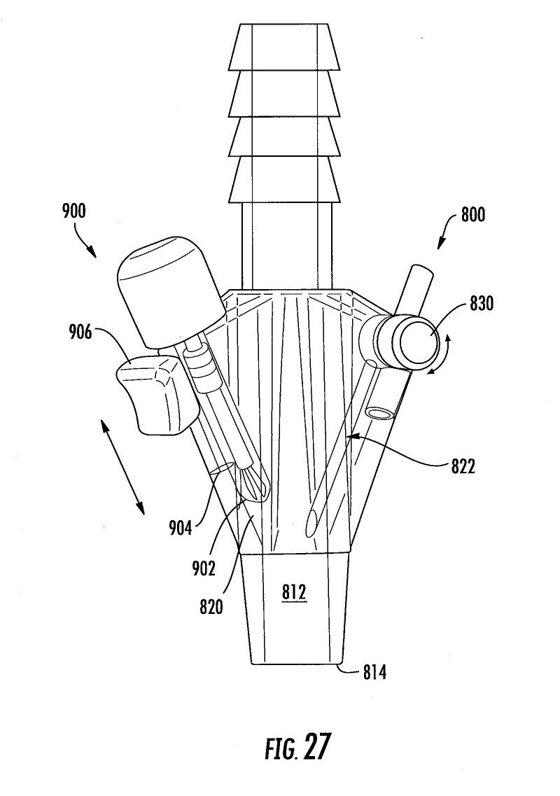

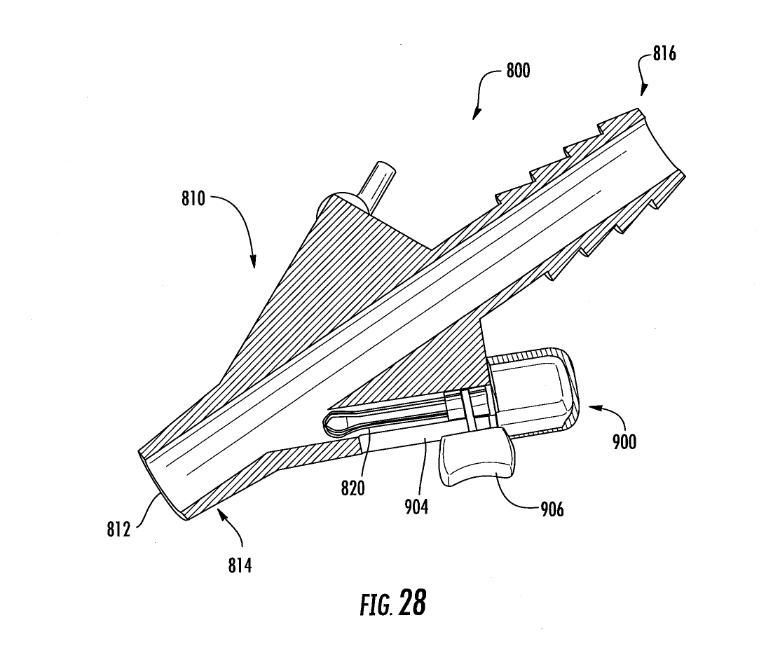

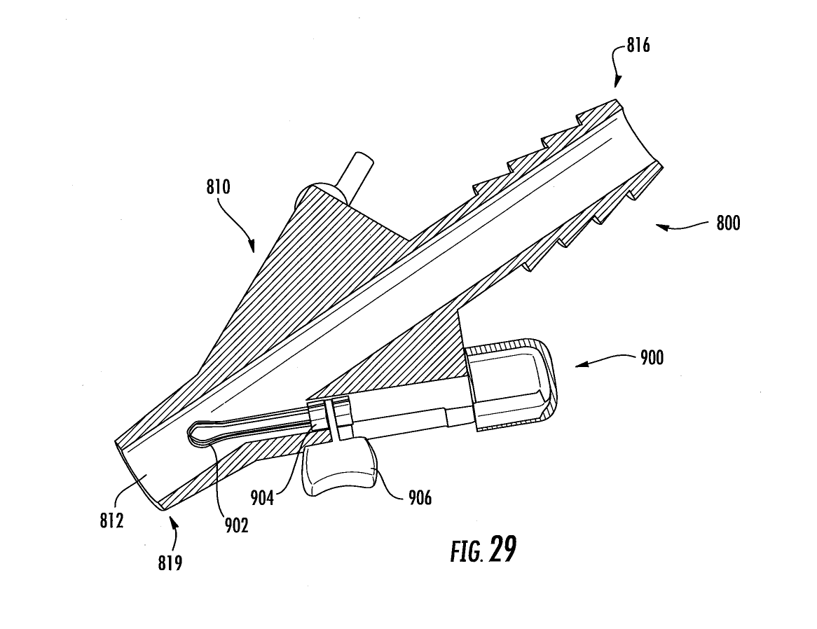

Although embodiments according to the invention are illustrated with respect to a subdural drainage housing 110/610 and drainage passageway 112/612 that is generally coaxially arranged with the blockage removal unit 200/700, it should be understood that other configurations may be used. For example, as illustrated in FIGS. 23-27, a subdural drainage device 800 includes a side-mounted blockage removal unit 900. The device 800 has a housing 810 with a drainage passageway 812 with a lower opening 814 configured to connect to a subdural space 22 of a skull 20, and an upper opening 816 that is configured to connect to a reservoir or tube. The housing 810 includes passageways 820, 822 for the blockage removal unit 900 and a drug delivery valve 830. The blockage removal unit 900 includes an end portion or deformable member 902, a telescoping body 904 and a handle 906. As illustrated, e.g., in FIGS. 28-29, the blockage removal unit 900 is movable between a retracted position in the passageway 820 in which the deformable member 902 is in the housing (FIG. 28), and an extended position in which the handle 906 is depressed so that the telescoping body 904 moves the deformable member 902 into the passageway 812 (FIG. 29) and out of the end 814 so that the deformable member 902 may dislodge, reduce or remove any blockages in the drainage passageway 812. However, in some embodiments, the deformable member 902 may extend into the subdural space and outside of the housing 810 in the extended position. The valve 830 is rotatable between an open and a closed position to accept a drug delivered therein.

In some embodiments, the interior diameter of the subdural drainage device or drainage passageway is about 4-5 mm, and the diameter of the drug delivery ports is about 1-2 mm, although any suitable dimensions may be used. The devices described herein may be rigid or somewhat flexible, and may be formed of any suitable polymeric, metallic, composite material or combination thereof. Although the devices described herein are generally linear, it should be understood that various elements may take different forms and shapes. For example, the proximal and/or distal end portions of the device housing or the blockage removal unit may be turned, angled, enlarged, and/or shaped, for example, to accommodate a lower profile.

Drains and reservoirs used to carry out the present invention may be any suitable form, including electromechanical devices that apply a negative pressure, simple tube and bulb devices, etc., and may be connected to the subdural drainage system at any suitable position, such as the housing, the subdural drainage device, catheter, catheter support or subdural drainage port. The housing according to embodiments of the present invention may be configured to attach to existing subdural drainage devices, such as a subdural evacuation port system (or “SEPS”) available from Medtronic, Inc., Minneapolis, Minn. In some embodiments, drains and/or reservoirs may connect to the external port of the subdural drainage systems described herein in a positive manner that maintains a sterile seal of the device, in like manner as described above.

Kits of the present invention can include the entire device, assembled or disassembled into components. The device or components thereof may be sealed, together or individually, in sterile packages or containers by any suitable technique. Where components are sealed into two or more separate sterile packages, those separate packages may in turn be packaged together in a larger package that contains multiple components. Printed instructions for use of the system may be included within and/or printed on the packaging, as desired.

The foregoing is illustrative of the present invention, and is not to be construed as limiting thereof. The invention is defined by the following claims, with equivalents of the claims to be included therein.

That which is claimed is:

1. A subdural drainage device, comprising:
a subdural drainage housing defining a drainage passageway, the drainage passageway having a lower opening configured to connect to a subdural space of a patient and an upper opening;
a blockage removal unit in the housing, the blockage removal unit having an end portion that is configured to reduce or remove blockages adjacent the lower opening of the drainage passageway and being movable between a retracted position in which the end portion is in the subdural drainage housing and an extended position in which the end portion of the blockage removal unit extends away from the subdural drainage housing via the lower opening of the drainage passageway; and
at least one side delivery port in the housing and in fluid communication with the drainage passageway, the at least one side delivery port being configured to receive a delivery device so that the delivery device delivers a therapeutic agent to the drainage passageway via the at least one side delivery port.

2. The subdural drainage device of claim 1, wherein the side delivery port comprises a port passageway having an outer opening that is configured to receive a delivery device and an inner opening that connects to the drainage passageway.

3. The subdural drainage device of claim 2, wherein the outer opening comprises a resealable member configured to be punctured by a delivery device comprising a needle and to be resealed when the needle is removed from the resealable member.

4. The subdural drainage device of claim 1, wherein the end portion of the blockage removal unit that is configured to reduce or remove blockages comprises a deformable member that is configured to move between a compact configuration when the end portion is in the subdural drainage housing in the retracted position and an expanded position when the end portion extends away from the subdural drainage housing via the lower opening of the drainage passageway in the extended position.

5. The subdural drainage device of claim 1, wherein the blockage removal unit is coaxial with the drainage passageway.

6. The subdural drainage device of claim 5, wherein the blockage removal unit comprises a drainage passageway therethrough.

7. The subdural drainage device of claim 5, wherein the blockage removal unit comprises a biasing member configured to maintain the blockage removal unit in the retracted position.

8. The subdural drainage device of claim 5, further comprising a locking member configured to move between a locked position in which a portion of the locking member abuts a portion of the blockage removal unit to maintain the blockage removal unit in the retracted position and an unlocked position in which the locking member releases the blockage removal unit so that the blockage removal unit may be moved to the extended position.

9. The subdural drainage device of claim 1, wherein the deformable member has a diameter that is greater than a diameter of the drainage passageway when the deformable member is in the extended position.

10. The subdural drainage device of claim 1, wherein the subdural drainage housing provides a sealed enclosure when the subdural drainage housing is connected to a skull of a subject.

11. The subdural drainage device of claim 1, wherein the blockage removal unit is mounted in a side of the housing and in communication with the drainage passageway.

12. The subdural drainage device of claim 1, further comprising first and second sealing members in the drainage passageway defining a sealing chamber.

13. The subdural drainage device of claim 12, wherein the first sealing member is mounted on the housing, and the second sealing member is mounted on the blockage removal unit such that when the second sealing member moves toward the first sealing member when the blockage removal unit is moved from the retracted position to the extended position to thereby increase or maintain sterility of the fluid drainage passageway.

14. A kit comprising a device of claim 1.

15. A kit of claim 12, wherein device is sealed in one or more a sterile packages or containers.

16. A method for draining the subdural space, said method comprising:
positioning a subdural drainage system on a patient, the subdural drainage system comprising:
a subdural drainage housing defining a drainage passageway, the drainage passageway having a lower opening configured to connect to a subdural space of a patient and an upper opening;
a blockage removal unit in the housing, the blockage removal unit having an end portion that is configured to reduce or remove blockages adjacent the lower opening of the drainage passageway and being movable between a retracted position in which the end portion is in the subdural drainage housing and an extended position in which the end portion of the blockage removal unit extends away from the subdural drainage housing via the lower opening of the drainage passageway; and
at least one side delivery port in the housing and in fluid communication with the drainage passageway, the at least one side delivery port being configured to receive a delivery device so that the delivery device delivers a therapeutic agent to the drainage passageway via the at least one side delivery port; and
moving the blockage removal unit between the retracted and extended positions so that the deformable member automatically extends to an extended, deformed position in which blockages are disrupted in and adjacent the drainage passageway.
